(19) United States
(12) Patent Application Publication
(10) Pub. No.: US 2014/0274733 A1
(43) Pub. Date: Sep. 18, 2014

(54) Methods and Systems for Local Sequence Alignment

(71) Applicant: LIFE TECHNOLOGIES CORPORATION, Carlsbad, CA (US)

(72) Inventors: Christian Koller, San Francisco, CA (US); Zheng ZHANG, Arcadia, CA (US)

(73) Assignee: LIFE TECHNOLOGIES CORPORATION, Carlsbad, CA (US)

(21) Appl. No.: 14/205,492

(22) Filed: Mar. 12, 2014

Related U.S. Application Data
(60) Provisional application No. 61/778,130, filed on Mar. 12, 2013.

(51) Int. Cl. C12Q 1/68

(52) U.S. Cl. CPC C12Q 1/6874
USPC 506/2; 506/38

(57) ABSTRACT

A method for nucleic acid sequencing includes: (a) disposing a plurality of template polynucleotide strands in a plurality of defined spaces disposed on a sensor array, at least some of the template polynucleotide strands having a sequencing primer and a polymerase operably bound therewith; (b) exposing the template polynucleotide strands with the sequencing primer and a polymerase operably bound therewith to a series of flows of nucleotide species flowed according to a predetermined ordering; (c) determining sequence information for a plurality of the template polynucleotide strands in the defined spaces based on the flows of nucleotide species to generate a plurality of sequencing reads corresponding to the template polynucleotide strands; and (d) aligning the plurality of sequencing reads using an alignment process comprising a first set of alignment criteria or penalties that are based on biological changes in sequence and a second set of alignment criteria or penalties that are based on a sequencing error mode.

### RELATED APPLICATIONS

This application is related to U.S. Provisional Application No. 61/778,130 filed Mar. 12, 2013, which is incorporated herein by reference in its entirety.

### FIELD

The present disclosure generally relates to the field of nucleic acid sequencing including systems and methods for local sequence alignment.

### INTRODUCTION

Upon completion of the Human Genome Project, one focus of the sequencing industry has shifted to finding higher throughput and/or lower cost nucleic acid sequencing technologies, sometimes referred to as “next generation” sequencing (NGS) technologies. In making sequencing higher throughput and/or less expensive, the goal is to make the technology more accessible. These goals can be reached through the use of sequencing platforms and methods that provide sample preparation for samples of significant complexity, sequencing larger numbers of samples in parallel (for example through use of barcodes and multiplex analysis), and/or processing high volumes of information efficiently and completing the analysis in a timely manner. Various methods, such as, for example, sequencing by synthesis, sequencing by hybridization, and sequencing by ligation are evolving to meet these challenges.

Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

Exemplary applications of NGS technologies include, but are not limited to: genomic variant detection, such as insertions/deletions, copy number variations, single nucleotide polymorphisms, etc., genomic resequencing, gene expression analysis and genomic profiling.

Accordingly, there is a need for further data analysis methods and systems that can efficiently process and analyze large volumes of data relating to nucleic acid sequence analysis and more particularly, to align or map nucleic acid fragments or sequences of various lengths. Further, there is a need for new data analysis methods and systems that can efficiently process data and signals indicative of electronically-detected chemical reactions, for example, nucleotide incorporation events, and transform these signals into other data and information, for example, base calls and nucleic acid sequence information and reads, which then can be aligned, for example, against a reference genome.

### SUMMARY

In light of the foregoing, the present teachings provide new and improved methods and systems for nucleic acid sequence analysis that can address and analyze data reflective of electronically-detected chemical targets and/or reaction by-products associated with nucleotide incorporation events without the need for exogenous labels or dyes to characterize nucleic acid sequences of interest. In various embodiments, the present teachings describe methods and systems that can process such data and various forms thereof including nucleotide flow orders to align or map fragments of the nucleic acid(s) of interest. These methodologies also can be applied to conventional sequencing techniques and in particular, sequencing by synthesis techniques.

In various embodiments, the present teachings describe a method of aligning a putative nucleic acid sequence or fragment of a sample nucleic acid template or complement thereof against a candidate reference nucleic acid sequence.

Numerous embodiments of the present teachings include a computer-useable medium having computer readable instructions stored thereon for execution by a processor to perform the various methods described herein.

The methods also can include transmitting, displaying, storing, or printing; or outputting to a user interface device, a computer readable storage medium, a local computer system or a remote computer system, information related to one or more of the alignments and the information associated with the alignments, such as the sample nucleic acid template, the signals, the defined space, the matrices, and equivalents thereof.

The present teachings also include a computer-useable medium having computer readable instructions stored thereon for execution by a processor to perform various embodiments of methods of the present teachings. It should be understood that the signals described herein generally refer to non-transitory signals, for example, an electronic signal, unless understood otherwise from the context of the discussion.

In various embodiments of systems of the present teachings for nucleic acid sequence analysis, a aligner module can be configured to practice and/or carry out various methods of the present and/or teachings as described herein and as understood by a skilled artisan.

It is to be understood that both the foregoing general description and the following detailed description are exemplary and explanatory only and are not intended to limit the scope of the present teachings.

### DRAWINGS

For a more complete understanding of the principles disclosed herein, and the advantages thereof, reference is now made to the following descriptions taken in conjunction with the accompanying drawings, in which:

FIG. 1 is a block diagram that illustrates an exemplary computer system, in accordance with various embodiments.

FIG. 2 is a schematic diagram of an exemplary system for reconstructing a nucleic acid sequence, in accordance with various embodiments.

FIG. 3 is a schematic diagram of an exemplary genetic analysis system, in accordance with various embodiments.

FIG. 4 is an exemplary diagram showing the sources of apparent variants, in accordance with various embodiments.

FIG. 5 is a flow diagram illustrating an exemplary method of aligning sequence reads to a reference sequence, in accordance with various embodiments.

FIG. 6 is a flow diagram illustrating an exemplary method of identifying variants, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of systems and methods for mapping and aligning sequence reads and identifying sequence variants are described herein.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless described otherwise, all technical and scientific terms used herein have a meaning as is commonly understood by one of ordinary skill in the art to which the various embodiments described herein belongs.

In various aspects of the present disclosure, a method for nucleic acid sequencing can include (a) disposing a plurality of template polynucleotide strands in a plurality of defined spaces disposed on a sensor array, at least some of the template polynucleotide strands having a sequencing primer and a polymerase operably bound therewith, (b) exposing the template polynucleotide strands with the sequencing primer and a polymerase operably bound therewith to a series of flows of nucleotide species flowed according to a predetermined ordering, and (c) determining sequence information for a plurality of the template polynucleotide strands in the defined spaces based on the flows of nucleotide species to generate a plurality of sequencing reads corresponding to the template polynucleotide strands. The method can further include (d) aligning the plurality of sequencing reads using an alignment process comprising a first set of alignment criteria or penalties that are based on biological changes in sequence and a second set of alignment criteria or penalties that are based on a sequencing error mode.

In various aspects of the present disclosure, a non-transitory machine-readable storage medium can comprise instructions which, when executed by a processor, can cause the processor to perform a method for nucleic acid sequencing including (a) disposing a plurality of template polynucleotide strands in a plurality of defined spaces disposed on a sensor array, at least some of the template polynucleotide strands having a sequencing primer and a polymerase operably bound therewith, (b) exposing the template polynucleotide strands with the sequencing primer and a polymerase operably bound therewith to a series of flows of nucleotide species flowed according to a predetermined ordering, and (c) determining sequence information for a plurality of the template polynucleotide strands in the defined spaces based on the flows of nucleotide species to generate a plurality of sequencing reads corresponding to the template polynucleotide strands. The method can further include (d) aligning the plurality of sequencing reads using an alignment process comprising a first set of alignment criteria or penalties that are based on biological changes in sequence and a second set of alignment criteria or penalties that are based on a sequencing error mode.

In various aspects of the present disclosure, a system can include a machine-readable memory and a processor. The processor can be configured to execute machine-readable instructions, which, when executed by the processor, can cause the system to perform a method for nucleic acid sequencing including (a) disposing a plurality of template polynucleotide strands in a plurality of defined spaces disposed on a sensor array, at least some of the template polynucleotide strands having a sequencing primer and a polymerase operably bound therewith, (b) exposing the template polynucleotide strands with the sequencing primer and a polymerase operably bound therewith to a series of flows of nucleotide species flowed according to a predetermined ordering, and (c) determining sequence information for a plurality of the template polynucleotide strands in the defined spaces based on the flows of nucleotide species to generate a plurality of sequencing reads corresponding to the template polynucleotide strands. The method can further include (d) aligning the plurality of sequencing reads using an alignment process comprising a first set of alignment criteria or penalties that are based on biological changes in sequence and a second set of alignment criteria or penalties that are based on a sequencing error mode.

In various embodiments, the first set of alignment criteria or penalties can include criteria that credit matching bases and penalize inserted, deleted, or mismatched bases. In various embodiments, the first set of alignment criteria or penalties comprises criteria can be assigned on a per base level. In various embodiments, the first set of alignment criteria or penalties can include different penalties being assigned to single nucleotide permutations than to insertions or deletions. In various embodiments, the first set of alignment criteria or penalties can include an affine gap penalty used in which a larger penalty is imposed for the existence of a gap and a smaller penalty is imposed for every base the gap increases in length.

In various embodiments, the second set of alignment criteria or penalties comprises a penalty being decreased as a function of homopolymer length. In various embodiments, the second set of alignment criteria or penalties can include a penalty that depends on an absolute difference in the length of two homopolymers. In various embodiments, the second set of alignment criteria or penalties can include a penalty that depends on a relative difference in the length of two homopolymers. In various embodiments, the second set of alignment criteria or penalties can include a penalty being reduced for sequence changes that do not shift flows at which subsequent homoploymers incorporate given the predetermined ordering.

It will be appreciated that there is an implied “about” prior to the temperatures, concentrations, times, number of bases, coverage, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

As used herein, “a” or “an” also may refer to “at least one” or “one or more.” Also, the use of “or” is inclusive, such that the phrase “A or B” is true when “A” is true, “B” is true, or both “A” and “B” are true.

Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

A “system” sets forth a set of components, real or abstract, comprising a whole where each component interacts with or is related to at least one other component within the whole.

A “biomolecule” may refer to any molecule that is produced by a biological organism, including large polymeric molecules such as proteins, polysaccharides, lipids, and nucleic acids (DNA and RNA) as well as small molecules such as primary metabolites, secondary metabolites, and other natural products.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the Personal Genome Machine (PGM) of Life Technologies Corp. provides massively parallel sequencing with enhanced accuracy. The PGM System and associated workflows, protocols, chemistries, etc. are described in more detail in U.S. Patent Application Publication No. 2009/0127589 and No. 2009/0026082, the entirety of each of these applications being incorporated herein by reference.

The phrase “sequencing run” refers to any step or portion of a sequencing experiment performed to determine some information relating to at least one biomolecule (e.g., nucleic acid molecule).

The phase “base space” refers to a representation of the sequence of nucleotides. The phase “flow space” refers to a representation of the incorporation event or non-incorporation event for a particular nucleotide flow. For example, flow space can be a series of values representing a nucleotide incorporation events (such as a one, “1”) or a non-incorporation event (such as a zero, “0”) for that particular nucleotide flow. Nucleotide flows having a non-incorporation event can be referred to as empty flows, and nucleotide flows having a nucleotide incorporation event can be referred to as positive flows. It should be understood that zeros and ones are convenient representations of a non-incorporation event and a nucleotide incorporation event; however, any other symbol or designation could be used alternatively to represent and/or identify these events and non-events. In particular, when multiple nucleotides are incorporated at a given position, such as for a homopolymer stretch, the value can be proportional to the number of nucleotide incorporation events and thus the length of the homopolymer stretch.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, a “somatic variation” or “somatic mutation” can refer to a variation in genetic sequence that results from a mutation that occurs in a non-germline cell. The variation can be passed on to daughter cells through mitotic division. This can result in a group of cells having a genetic difference from the rest of the cells of an organism. Additionally, as the variation does not occur in a germline cell, the mutation may not be inherited by progeny organisms.

### Computer-Implemented System

FIG. 1 is a block diagram that illustrates a computer system 100, upon which embodiments of the present teachings may be implemented. In various embodiments, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining base calls, and instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, processor 104 can include a plurality of logic gates. The logic gates can include AND gates, OR gates, NOT gates, NAND gates, NOR gates, EXOR gates, EXNOR gates, or any combination thereof. An AND gate can produce a high output only if all the inputs are high. An OR gate can produce a high output if one or more of the inputs are high. A NOT gate can produce an inverted version of the input as an output, such as outputting a high value when the input is low. A NAND (NOT-AND) gate can produce an inverted AND output, such that the output will be high if any of the inputs are low. A NOR (NOT-OR) gate can produce an inverted OR output, such that the NOR gate output is low if any of the inputs are high. An EXOR (Exclusive-OR) gate can produce a high output if either, but not both, inputs are high. An EXNOR (Exclusive-NOR) gate can produce an inverted EXOR output, such that the output is low if either, but not both, inputs are high.

TABLE 1
Logic Gates Truth Table

| INPUTS | | OUTPUTS | | | | | | |
|---|---|---|---|---|---|---|---|---|
| A | B | NOT A | AND | NAND | OR | NOR | EXOR | EXNOR |
| 0 | 0 | 1 | 0 | 1 | 0 | 1 | 0 | 1 |
| 0 | 1 | 1 | 0 | 1 | 1 | 0 | 1 | 0 |
| 1 | 0 | 0 | 0 | 1 | 1 | 0 | 1 | 0 |
| 1 | 1 | 0 | 1 | 0 | 1 | 0 | 0 | 1 |

One of skill in the art would appreciate that the logic gates can be used in various combinations to perform comparisons, arithmetic operations, and the like. Further, one of skill in the art would appreciate how to sequence the use of various combinations of logic gates to perform complex processes, such as the processes described herein.

In an example, a 1-bit binary comparison can be performed using a XNOR gate since the result is high only when the two inputs are the same. A comparison of two multi-bit values can be performed by using multiple XNOR gates to compare each pair of bits, and the combining the output of the XNOR gates using and AND gates, such that the result can be true only when each pair of bits have the same value. If any pair of bits does not have the same value, the result of the corresponding XNOR gate can be low, and the output of the AND gate receiving the low input can be low.

In another example, a 1-bit adder can be implemented using a combination of AND gates and XOR gates. Specifically, the 1-bit adder can receive three inputs, the two bits to be added (A and B) and a carry bit (Cin), and two outputs, the sum (S) and a carry out bit (Cout). The Cin bit can be set to 0 for addition of two one bit values, or can be used to couple multiple 1-bit adders together to add two multi-bit values by receiving the Cout from a lower order adder. In an exemplary embodiment, S can be implemented by applying the A and B inputs to a XOR gate, and then applying the result and Cin to another XOR gate. Cout can be implemented by applying the A and B inputs to an AND gate, the result of the A-B XOR from the SUM and the Cin to another AND, and applying the input of the AND gates to a XOR gate.

TABLE 2
1-bit Adder Truth Table

| | |
|---|---|
| INPUTS | OUTPUTS |

| A | B | Cin | S | Cout |
|---|---|---|---|---|
| 0 | 0 | 0 | 0 | 0 |
| 1 | 0 | 0 | 0 | 1 |
| 0 | 1 | 0 | 0 | 1 |
| 1 | 1 | 0 | 1 | 0 |
| 0 | 0 | 1 | 0 | 1 |
| 1 | 0 | 1 | 1 | 0 |
| 0 | 1 | 1 | 1 | 0 |
| 1 | 1 | 1 | 1 | 1 |

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results can be provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 can cause processor 104 to perform the processes described herein. In various embodiments, instructions in the memory can sequence the use of various combinations of logic gates available within the processor to perform the processes describe herein. Alternatively hard-wired circuitry can be used in place of or in combination with software instructions to implement the present teachings. In various embodiments, the hard-wired circuitry can include the necessary logic gates, operated in the necessary sequence to perform the processes described herein. Thus implementations of the present teachings are not limited to any specific combination of hardware circuitry and software.

The term “computer-readable medium” as used herein refers to any media that participates in providing instructions to processor 104 for execution. Such a medium can take many forms, including but not limited to, non-volatile media, volatile media, and transmission media. Examples of non-volatile media can include, but are not limited to, optical or magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of non-transitory computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

### Nucleic Acid Sequencing Platforms

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

Various embodiments of nucleic acid sequencing platforms, such as a nucleic acid sequencer, can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Pat. No. 7,948,015, U.S. Patent Application Publication No. 2010/0137143, No. 2009/0026082, and 2010/0282617, which are all incorporated by reference herein in their entirety. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 206 can include an excitation system to cause a probe, such as a fluorescent dye, to emit a signal. The excitation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 2009/0325145, the entirety of which being incorporated herein by reference) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

### System and Methods for Identifying Sequence Variation

FIG. 3 is a schematic diagram of a system for identifying variants, in accordance with various embodiments.

As depicted herein, variant analysis system 300 can include a nucleic acid sequence analysis device 304 (e.g., nucleic acid sequencer, real-time/digital/quantitative PCR instrument, microarray scanner, etc.), an analytics computing server/node/device 302, and a display 310 and/or a client device terminal 308.

In various embodiments, the analytics computing sever/node/device 302 can be communicatively connected to the nucleic acid sequence analysis device 304, and client device terminal 308 via a network connection 324 that can be either a “hardwired” physical network connection (e.g., Internet, LAN, WAN, VPN, etc.) or a wireless network connection (e.g., Wi-Fi, WLAN, etc.).

In various embodiments, the analytics computing device/server/node 302 can be a workstation, mainframe computer, distributed computing node (part of a “cloud computing” or distributed networking system), personal computer, mobile device, etc. In various embodiments, the nucleic acid sequence analysis device 304 can be a nucleic acid sequencer, real-time/digital/quantitative PCR instrument, microarray scanner, etc. It should be understood, however, that the nucleic acid sequence analysis device 304 can essentially be any type of instrument that can generate nucleic acid sequence data from samples obtained from an individual.

The analytics computing server/node/device 302 can be configured to host an optional pre-processing module 312, a mapping module 314, and a variant calling module 316.

Pre-processing module 312 can be configured to receive from the nucleic acid sequence analysis device 304 and perform processing steps, such as conversion from flow space to base space, determining call quality values, preparing the read data for use by the mapping module 314, and the like.

The mapping module 314 can be configured to align (i.e., map) a nucleic acid sequence read to a reference sequence. Generally, the length of the sequence read is substantially less than the length of the reference sequence. In reference sequence mapping/alignment, sequence reads are assembled against an existing backbone sequence (e.g., reference sequence, etc.) to build a sequence that is similar but not necessarily identical to the backbone sequence. Once a backbone sequence is found for an organism, comparative sequencing or re-sequencing can be used to characterize the genetic diversity within the organism's species or between closely related species. In various embodiments, the reference sequence can be a whole/partial genome, whole/partial exome, etc. Alignment features relating to the present disclosure may comprise one or more features described in Homer, U.S. Pat. Appl. Publ. No. 2012/0197623, and Utiramerur et al., U.S. patent application Ser. No. 13/787,221, which are all incorporated by reference herein in their entirety.

In various embodiments, the sequence read and reference sequence can be represented as a sequence of nucleotide base symbols in base space. In various embodiments, the sequence read and reference sequence can be represented as one or more colors in color space. In various embodiments, the sequence read and reference sequence can be represented as nucleotide base symbols with signal or numerical quantitation components in flow space.

In various embodiments, the alignment of the sequence fragment and reference sequence can include a limited number of mismatches between the bases that comprise the sequence fragment and the bases that comprise the reference sequence. Generally, the sequence fragment can be aligned to a portion of the reference sequence in order to minimize the number of mismatches between the sequence fragment and the reference sequence.

The variant calling module 316 can include a realignment engine 318, a variant calling engine 320, and an optional post processing engine 322. In various embodiments, variant calling module 316 can be in communications with the mapping module 314. That is, the variant calling module 316 can request and receive data and information (through, e.g., data streams, data files, text files, etc.) from mapping module 314. In various embodiments, the variant calling module 316 can be configured to communicate variants called for a sample genome as a *.vcf, *.gff, or *.hdf data file. It should be understood, however, that the called variants can be communicated using any file format as long as the called variant information can be parsed and/or extracted for later processing/analysis.

The realignment engine 318 can be configured to receive mapped reads from the mapping module 314, realign the mapped reads in flow space, and provide the flow space alignments to the variant calling engine 320. In various embodiments, the mapped read can be realigned to the reference sequence using a local sequence aligning method, for example, a Smith-Waterman algorithm (see, e.g., Smith and Waterman, Journal of Molecular Biology 147(10:195-197 (1981)). The resulting alignments can be aggregated to determine the best mapping(s) or goodness of fit. In particular embodiments, the realignment can utilize context dependent penalties for gaps and mismatches.

The variant calling engine 320 can be configured to receive flow space information from the realignment engine 318 and identify differences between the aligned reads and the reference sequence. In various embodiments, the variant calling engine can evaluate potential variants to determine a likelihood that variant is true and not a result of a sequencing error. The evaluation can involve reevaluation of the flow space information for the reads aligned to the position for evidence of the potential variant, statistical analysis of the support for the variant from multiple reads aligned to the same position, and the like.

Post processing engine 322 can be configured to receive the variants identified by the variant calling engine 320 and perform additional processing steps, such as conversion from flow space to base space, filtering adjacent variants, and formatting the variant data for display on display 310 or use by client device 308. Examples of filters that the post-processing engine 322 may apply include a minimum score threshold, a minimum number of reads including the variant, a minimum frequency of reads including the variant, a minimum mapping quality, a strand probability, and region filtering.

Client device 308 can be a thin client or thick client computing device. In various embodiments, client terminal 308 can have a web browser (e.g., INTERNET EXPLORER™, FIREFOX™, SAFARI™, etc) that can be used to communicate information to and/or control the operation of the pre-processing module 312, mapping module 314, realignment engine 318, variant calling engine 320, and post processing engine 322 using a browser to control their function. For example, the client terminal 308 can be used to configure the operating parameters (e.g., match scoring parameters, annotations parameters, filtering parameters, data security and retention parameters, etc.) of the various modules, depending on the requirements of the particular application. Similarly, client terminal 308 can also be configure to display the results of the analysis performed by the variant calling module 316 and the nucleic acid sequencer 304.

It should be understood that the various data stores disclosed as part of system 300 can represent hardware-based storage devices (e.g., hard drive, flash memory, RAM, ROM, network attached storage, etc.) or instantiations of a database stored on a standalone or networked computing device(s).

It should also be appreciated that the various data stores and modules/engines shown as being part of the system 300 can be combined or collapsed into a single module/engine/data store, depending on the requirements of the particular application or system architecture. Moreover, in various embodiments, the system 300 can comprise additional modules, engines, components or data stores as needed by the particular application or system architecture.

In various embodiments, the system 300 can be configured to process the nucleic acid reads in color space. In various embodiments, system 300 can be configured to process the nucleic acid reads in base space. In various embodiments, system 300 can be configured to process the nucleic acid sequence reads in flow space. Data analysis aspects relating to the present disclosure (e.g., processing of measurements, calling of bases, etc.) may comprise one or more features described in Davey et al., U.S. Pat. Appl. Publ. No. 2012/0109598, and Sikora et al., U.S. patent application Ser. Nos. 13/588,408 and 13/645,058, which are all incorporated by reference entirety herein in their entirety. It should be understood, however, that the system 300 disclosed herein can process or analyze nucleic acid sequence data in any schema or format as long as the schema or format can convey the base identity and position of the nucleic acid sequence.

FIG. 4 is an exemplary diagram showing the sources of apparent variants, in accordance with various embodiments. The reference sequence can be illustrated at block 402. Biological changes, represented by block 404, can result in changes the sequence, represented by block 404. The biological changes can include single and multiple nucleotide polymorphism, insertions, deletions, rearrangements, and other changes. Various biological mechanisms are known to account for the biological changes, including replication errors, translocations, insertional mutations, etc. During the sequencing process, sequencing errors, represented by block 408, can be introduced into the reads, represented by block 410. There errors can be due to noise in the sequencing data, or errors due to misincorporations. Generally, biological changes can be observed in a large number of reads, whereas sequencing errors can be isolated to a small number of reads.

FIG. 5 is an exemplary flow diagram showing a method 500 for aligning sequence reads to a reference sequence, in accordance with various embodiments. At 402, template polynucleotide strands can be applied to a sensor array. In various embodiments, the template strands can be applied to defined spaces of the sensor array. One or more template strands can be applied to a defined space, and generally, the template strands within a defined space can have a substantially identical nucleotide sequence. Additionally, sequencing primers and a nucleic acid polymerase can be applied to the defined spaces. In various embodiments, the template strands, sequencing primers and nucleic acid polymerase can form a nucleic acid synthesis complex.

At 404, the template stands, and the nucleic acid synthesis complex can be exposed to a series of flows of nucleotide species in a predetermined order. Flow ordering aspects relating to the present disclosure may comprise one or more features described in Hubbell et al., U.S. Pat. Appl. Publ. No. 2012/0264621, which is incorporated by reference herein in its entirety. In various embodiments, the nucleic acid synthesis complex can incorporate nucleotides from nucleotide flows that match the next base needed in the synthesis of a complementary strand. In particular embodiments, the incorporation can lead to a release of a hydrogen ion or other leaving group that can be detected by the sensor. The amount of the leaving group detectable by the sensor can be proportional to the number of incorporations, such as when two consecutive identical nucleotides are incorporated, the amount of the leaving group can be twice as great as the amount of leaving group when only a single nucleotide is incorporated. When the nucleotide flow does not match the next nucleotide needed for synthesis of the complementary strand, a nucleotide may not be incorporated and therefore no leaving group is released for the sensor to detect.

At 506, sequencing information can be determined for the template polynucleotide stands to generate sequence reads for the template stands. The sequencing information can include flow information, such as a signal recorded for the polynucleotide stand for each of the predefined nucleotide flows, a putative base sequence of the template or complementary stand, or any combination thereof.

At 508, the sequence reads can be aligned to a reference sequence. In various embodiments, the alignment process can include a set of alignment criteria or penalties based on biological changes and a set of alignment criteria or penalties based on sequencing error modes. Alignment features relating to the present disclosure may comprise one or more features described in Homer, U.S. Pat. Appl. Publ. No. 2012/0197623, and Utiramerur et al., U.S. patent application Ser. No. 13/787,221, which are all incorporated by reference herein in their entirety.

In various embodiments, the alignment process can involve a dynamic programming algorithm, such as a Smith-Waterman algorithm. The algorithm may apply credits for matching bases and penalties for inserted, deleted, or mismatched bases. In various embodiments, the criteria or penalties can be on a per base level. The penalties may include penalties for initiating a gap (insertion or deletion) and extending a gap. The penalty for initiation a gap (penalty for a gap to exist) may be greater than the penalty imported for every additional base in the gap. Further, penalties assigned for mismatches may be different than penalties assigned for an insertion or deletion.

Further, the penalties associated with sequencing errors may include a penalty for a difference in homopolymer length between the read and the reference. The homopolymer length penalty may decrease as a function of homopolymer length, such that a difference in a homopolymer length for a dimer (homopolymer length of 2) may be greater than the penalty when the homopolymer length is 7. The homopolymer length penalty can depend on the absolute difference in the length of the homopolymer in the read and the reference, or the penalty can depend on the relative difference. Further, the penalties associated with sequencing errors may include reduced penalties for sequencing changes that do not shift flows at which subsequent homopolymers are incorporated given the predetermined ordering. Erroneous calls (sequencing errors) may not influence the flows in which subsequent bases are incorporated. For example, an undercall of a T homopolymer may not change the flows in which subsequence bases are incorporated. In contrast, a biological change incorporating an A between two Ts could alter the flows in which subsequence bases are incorporated.

In various embodiments, the penalty applied for a mismatch at a given position in the sequence can depend on the type of mismatch (insertion/deletion vs. alternate base) as well as the sequence or flow space context.

FIG. 6 is an exemplary flow diagram showing a method 600 for aligning identifying variants based on a plurality of sequence reads, in accordance with various embodiments. At 602, the sequence information can be obtained. At 604, the reads can be mapped to a reference sequence. The reads can be mapped using various mapping algorithms known in the art. At 606, the reads can be realigned to the reference sequence. Specifically, the alignment algorithm previously described can optimize the alignment of the read to the reference operating on the local reference sequence, as opposed to the mapping algorithm which may be optimized to find the closest matching location rather than an optimal alignment at a particular location. In various embodiments, the mapping algorithm may identify a partial alignment at a location, and the realignment algorithm can identify an extended alignment of the read to the reference sequence. In various embodiments, the realignment can be used on reads where there are a significant number of mismatches between the read and the reference or where there are stretches of aligned sequence with multiple errors. In other embodiments, the realignment algorithm can be applied to all reads.

At 608, variants between the target sequence and the reference sequence can be identified by comparison of multiple reads aligned at the same location of the reference sequence. Generally, multiple reads containing the variant provide stronger evidence of a true variant than a single read containing the variant. Variant identification features relating to the present disclosure may comprise one or more features described in Hyland et al., Pat. Appl. Publ. No. 2013/0073214, Utiramerur et al., Pat. Appl. Publ. No. 2014/0052381, and Brinza et al., Pat. Appl. Publ. No. 2013/0345066, which are all incorporated by reference herein in their entirety.

In various embodiments, the methods of the present teachings may be implemented in a software program and applications written in conventional programming languages such as C, C++, etc.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

What is claimed is:

1. A method for nucleic acid sequencing, comprising:
(a) disposing a plurality of template polynucleotide strands in a plurality of defined spaces disposed on a sensor array, at least some of the template polynucleotide strands having a sequencing primer and a polymerase operably bound therewith;
(b) exposing the template polynucleotide strands with the sequencing primer and a polymerase operably bound therewith to a series of flows of nucleotide species flowed according to a predetermined ordering;
(c) determining sequence information for a plurality of the template polynucleotide strands in the defined spaces based on the flows of nucleotide species to generate a plurality of sequencing reads corresponding to the template polynucleotide strands; and
(d) aligning the plurality of sequencing reads using an alignment process comprising a first set of alignment criteria or penalties that are based on biological changes in sequence and a second set of alignment criteria or penalties that are based on a sequencing error mode.

2. The method of claim 1, wherein the first set of alignment criteria or penalties comprises criteria that credit matching bases and penalize inserted, deleted, or mismatched bases.

3. The method of claim 1, wherein the first set of alignment criteria or penalties comprises different penalties being assigned to single nucleotide permutations than to insertions or deletions.

4. The method of claim 1, wherein the first set of alignment criteria or penalties comprises an affine gap penalty used in which a larger penalty is imposed for the existence of a gap and a smaller penalty is imposed for every base the gap increases in length.

5. The method of claim 1, wherein the second set of alignment criteria or penalties comprises a penalty being decreased as a function of homopolymer length.

6. The method of claim 1, wherein the second set of alignment criteria or penalties comprises a penalty that depends on an absolute difference in the length of two homopolymers.

7. The method of claim 1, wherein the second set of alignment criteria or penalties comprises a penalty that depends on a relative difference in the length of two homopolymers.

8. The method of claim 1, wherein the second set of alignment criteria or penalties comprises a penalty being reduced for sequence changes that do not shift flows at which subsequent homoploymers incorporate given the predetermined ordering.

9. A non-transitory machine-readable storage medium comprising instructions which, when executed by a processor, cause the processor to perform a method for nucleic acid sequencing comprising:
(a) exposing a plurality of template polynucleotide disposed in a plurality of defined spaces disposed on a sensor array, at least some of the template polynucleotide strands having a sequencing primer and a polymerase operably bound therewith, to a series of flows of nucleotide species flowed according to a predetermined ordering;
(b) determining sequence information for a plurality of the template polynucleotide strands in the defined spaces based on the flows of nucleotide species to generate a plurality of sequencing reads corresponding to the template polynucleotide strands; and
(c) aligning the plurality of sequencing reads using an alignment process comprising a first set of alignment criteria or penalties that are based on biological changes in sequence and a second set of alignment criteria or penalties that are based on a sequencing error mode.

10. The non-transitory machine-readable storage medium of claim 9, wherein the first set of alignment criteria or penalties comprises criteria that credit matching bases and penalize inserted, deleted, or mismatched bases.

11. The non-transitory machine-readable storage medium of claim 9, wherein the first set of alignment criteria or penalties comprises criteria assigned on a per base level.

12. The non-transitory machine-readable storage medium of claim 9, wherein the first set of alignment criteria or penalties comprises different penalties being assigned to single nucleotide permutations than to insertions or deletions.

13. The non-transitory machine-readable storage medium of claim 9, wherein the first set of alignment criteria or penalties comprises an affine gap penalty used in which a larger penalty is imposed for the existence of a gap and a smaller penalty is imposed for every base the gap increases in length.

14. The non-transitory machine-readable storage medium of claim 9, wherein the second set of alignment criteria or penalties comprises a penalty being decreased as a function of homopolymer length.

15. The non-transitory machine-readable storage medium of claim 9, wherein the second set of alignment criteria or penalties comprises a penalty being reduced for sequence changes that do not shift flows at which subsequent homoploymers incorporate given the predetermined ordering.

16. A system, including:
a machine-readable memory; and
a processor configured to execute machine-readable instructions, which, when executed by the processor, cause the system to perform a method for nucleic acid sequencing, comprising:
(a) exposing a plurality of template polynucleotide disposed in a plurality of defined spaces disposed on a sensor array, at least some of the template polynucleotide strands having a sequencing primer and a polymerase operably bound therewith, to a series of flows of nucleotide species flowed according to a predetermined ordering;
(b) determining sequence information for a plurality of the template polynucleotide strands in the defined spaces based on the flows of nucleotide species to generate a plurality of sequencing reads corresponding to the template polynucleotide strands; and
(c) aligning the plurality of sequencing reads using an alignment process comprising a first set of alignment criteria or penalties that are based on biological changes in sequence and a second set of alignment criteria or penalties that are based on a sequencing error mode.

17. The system of claim 16, wherein the first set of alignment criteria or penalties comprises different penalties being assigned to single nucleotide permutations than to insertions or deletions.

18. The system of claim 16, wherein the first set of alignment criteria or penalties comprises an affine gap penalty used in which a larger penalty is imposed for the existence of a gap and a smaller penalty is imposed for every base the gap increases in length.

19. The system of claim 16, wherein the second set of alignment criteria or penalties comprises a penalty being decreased as a function of homopolymer length.

20. The system of claim 16, wherein the second set of alignment criteria or penalties comprises a penalty being reduced for sequence changes that do not shift flows at which subsequent homoploymers incorporate given the predetermined ordering.
